(19) United States
(12) Patent Application Publication
(10) Pub. No.: US 2005/0085635 A1
(43) Pub. Date: Apr. 21, 2005

(54) Method of mycophenolate mofetil preparation

(76) Inventors: Miloslav Chudik, Karvina-Mizerov (CZ); Ales Husek, Opava (CZ)

Correspondence Address: IVAX CORPORATION, 4400 BISCAYNE BOULEVARD, MIAMI, FL, 33137, US

(21) Appl. No.: 10/480,058

(22) PCT Filed: Jun. 8, 2002
(86) PCT No.: PCT/US02/18274

(30) Foreign Application Priority Data
Jun. 8, 2001 (CZ) PV2001-2071

(51) Int. Cl. C07D 413/02

(52) U.S. Cl. 544/147

(57) ABSTRACT

Synthesis of mycophenolate mofetil (1), where R¹=2-(-morpholinyl)ethyl and R²=hydrogen atom, includes reaction of mycophenolic acid with 4-(2-hydroxyethyl)morpholine in a suitable solvent under azeotropic separation of water.

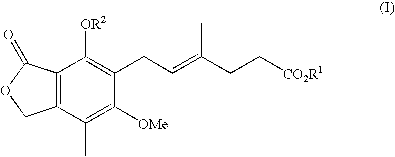

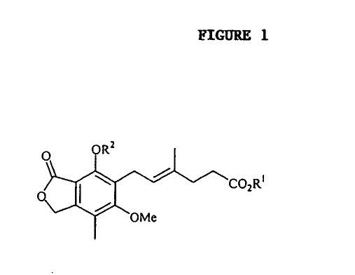

### FIELD OF THE INVENTION

This invention refers to method of mycophenolate mofetil preparation according to the formula I 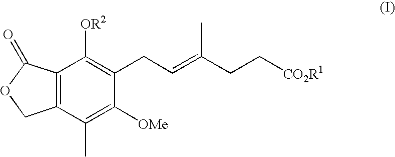
where
R¹ is 2-(4-morpholinyl)ethyl,
R² is hydrogen atom.

Mycophenolate mofetil (I) is used as an immunosuppressive for prophylactic treatment in combination with other immunosuppressives (cyclosporine A, prednisone), or for treatment of refractory rejections in patients after renal transplantation. Chemically, mycophenolate mofetil is 2-(4-morpholinyl)ethyl ester of mycophenolic acid (R¹=R²=H), which has cytostatic effect. It carries out inosine monophosphate dehydrogenaze selective inhibition, and this way also de novo synthesis pathway of guanosine nucleotides and their incorporation into DNK. This way cytostatic effect to lymphocytes is higher that to other cells.

### BACKGROUND ART

Synthesis of mycophenolate mofetil in accordance with the formula I (R¹=2-morpholinoethyl, R²═H) is described in the basic patent EP 281 713 B1 (1987) and several other patents: U.S. Pat. No. 4,808,592 (1989), U.S. Pat. No. 4,753,935 (1988), U.S. Pat. No. 4,952,579 (1990), U.S. Pat. No. 4,984,793 (1990), U.S. Pat. No. 4,786,637 (1988). In accordance with these patents mycophenolate mofetil may be prepared using two standard esterification methods (see Synthetic Organic Chemistry, R. B. Wagner and H. D. Zook (Wiley, New York), 1956, pages 479 to 532): reaction of mycophenolic chloride with excessive amount of 2-morpholinoethanol and condensation using dicyclohexylcarbodiimide (DDC). Esterification via the acid chloride is based on reaction of excessive amount of 2-morpholinoethanol with mycophenolic acid chloride that has been prepared from mycophenolic acid using suitable chlorinating agent (thionylchloride, oxalylchloride etc.). Use of the excessive amount of 2-morpholinoethanol (up to 3 equivalents), formation of dimmers (about 2%, R¹=H or 2-morpholinoethyl, R²=mycophenolic acid) represents a disadvantage of the two-stage process, there are also problems with colour of the product. Formation of unjustifiable amount of impurities and dicyclohexylurea that may be eliminated from the reaction mixture only by a chromatography is a disadvantage of DCC use as an activating agent.

The U.S. Pat. No. 5,247,083 dated 1993 describes preparation of mycophenolate mofetil by reflux of mycophenolic acid and 2-morpholinoethanol in a suitable solvent or a mixture of solvents under azeotropic water separation. Dichloromethane, benzene, toluene, xylene and higher hydrocarbons are given in the claims and examples. The most suitable solvents are toluene, xylene and their mixture in proportion 1:1. A long reaction period necessary to reach sufficient conversion (depending on the solvent used about 60 to 100 hours) and colour of the product (light violet crystal) are the disadvantages of this method.

Object of the international application No. WO 00/34503 dated 2000 is mycophenolic acid esterification with 2-morpholinoethanol using enzyme catalysis. This way mycophenolate mofetil may be obtained in high yield and purity, however, the method may not be used in industry. Within this patent method of mycophenolic acid esterification by boiling in 2-morpholinoethanol without any solvent is described but considering price of 2-morpholinoethanol the method is not suitable either.

### DISCLOSURE OF THE INVENTION

It was surprising during optimisation of mycophenolate mofetil preparation by mycophenolic acid direct esterification with 2-morpholinoethanol under azeotropic separation of water that thanks to use of dibutyl ether, unlike toluene or xylene, the reaction is slightly accelerated. Thanks to the use of higher ethers the problems with the colour of the product that had been monitored in toluene or xylene were eliminated. Low solubility of mycophenolate mofetil in higher ethers is also a favourable property as it makes product isolation from high-boiling solvent easier. That is why the proposed method represents the most favourable alternative to the method described under the patent U.S. Pat. No. 5,247,083.

Process in accordance with invention solves preparation of mycophenolate mofetil as follows:

Mycophenolic acid is esterified by reflux in ethers (general formula R³OR⁴, where R³, R⁴=alkyl, aryl), boiling point of which is 120° C. as minimum, under azeotropic separation of water and under use of excessive amount of 2-morpholinoethanol (1.01 to 3 molar equivalents). Reaction time is in the range 5 to 50 hours and reaction temperature is higher than 120° C. depending on the solvent used. The ratio mycophenolic acid: solvent used is in the range 1 g:2 ml to 1 g:5 ml. Conversion is in the range 80 to 98%. After raw product recrystallization mycophenolate mofetil is obtained with purity 99.0% as minimum and yield 70% as minimum.

### EXAMPLES

The invention is illustrated with the following examples that however do not limit extent of the patent in any way.

### Example 1

Mycophenolate mofetil; use of dibutyl ether as solvent

10 g mycophenolic acid were put in a reaction flask with a reflux cooler together with 20 ml dibutyl ether. Stirring vigorously the mixture was warmed up to the temperature of 50 to 60° C. and then 4 ml 2-morpholinoethanol were dropped in. The reaction mixture was warmed up to boiling under azeotropic separation of water. After 48 hours the mixture was cooled up to the laboratory temperature and diluted with 20 ml dichloromethane. The solution was extracted twice with 10 ml 0.5 M aqueous K₂CO₃ and once with 10 ml of water. Then dichloromethane was distilled off under vacuum and the suspension was cooled up to 10 to 15° C. Crystallized mycophenolate mofetil was removed by suction and recrystallized from ethyl acetate. After the removal by suction and drying the crystals 11 g (78%) mycophenolate mofetil was obtained with purity>99.0% (HPLC).

### Example 2

Mycophenolate mofetil; use of dipentyl ether as solvent

10 g mycophenolic acid were put in a reaction flask with a reflux cooler together with 20 ml dipentyl ether. Stirring vigorously the mixture was warmed up to the temperature of 50 to 60° C. and then 4 ml 2-morpholinoethanol were dropped in. The reaction mixture was warmed up to boiling under azeotropic separation of water. After 6 hours the mixture was cooled up to the laboratory temperature and diluted with 20 ml dichloromethane. The solution was extracted twice with 10 ml 0.5 M aqueous K₂CO₃ and once with 10 ml of water. Then dichloromethane was distilled off under vacuum and the suspension was cooled up to 10 to 15° C. Crystallized mycophenolate mofetil was removed by suction and recrystallized from ethyl acetate. After the removal by suction and drying the crystals 10 g (71%) mycophenolate mofetil was obtained with purity>99.0% (HPLC).

### Example 3

Mycophenolate mofetil; use excess of 2-morfpholinoethanol

10 g mycophenolic acid was put in a reaction flask with a reflux cooler together with 20 ml dibutyl ether. Stirring vigorously the mixture was warmed up to the temperature of 50 to 60° C. and then 4,8 ml 2-morpholinoethanol was added in. The reaction mixture was warmed up to boiling under azeotropic separation of water. After 15 hours the mixture was cooled up to the laboratory temperature and diluted with 25 ml dichloromethane. The solution was extracted twice with 10 ml of 1% aqueous ammonia and once with 10 ml of water. Then dichloromethane was distilled off under vacuum and the suspension was cooled up to 10 to 15° C. Crystallized mycophenolate mofetil was removed by suction and recrystallized from ethyl acetate. After the removal by suction and drying the crystals 11,1 g (82%) mycophenolate mofetil was obtained with purity>99.0% (HPLC).

1. The process of preparation of mycophenolate mofetil by direct esterification of mycophenolic acid and 2-morpholinoethanol characterized with esterification carried out under boiling in ethers.

2. The process according to claim 1, characterized with the use of ethers as solvent of the general formula R³OR⁴, where R³ and R⁴ are independently alkyl or aryl.

3. The process according to claim 2, characterized with the use of ethers as solvent of boiling point above 120° C.

4. The process according to claim 1, characterized with the use of 1.01 up to 3.0 molar equivalents of 2-morpholinoethanol.

5. The process according to claim 3, characterized with the use of dibutylether as an inert solvent.

6. The process according to claim 5, characterized with the starting temperature of the reaction ranging between 130° C. and 138° C. and the final temperature of the reaction ranging between 140° C. and 145° C.

7. The process according to claim 5, characterized with the reflux time ranging from 30 to 80 hours.

8. The process according to claim 5, characterized with the ratio of mycophenolic acid to dibutylether ranging from 1 g/2 ml to 1 g/5 ml.
